(19) United States
(12) Patent Application Publication
(10) Pub. No.: US 2025/0327142 A1
(43) Pub. Date: Oct. 23, 2025

(54) METHODS OF COLLECTING AND ANALYZING DUST SAMPLES FOR SURVEILLANCE OF VIRAL DISEASES

(71) Applicants: Ohio State Innovation Foundation, Columbus, OH (US); UNIVERSITY OF NOTRE DAME DU LAC, South Bend, IN (US)

(72) Inventors: Karen DANNEMILLER, Columbus, OH (US); Natalie HULL, Columbus, OH (US); Kyle BIBBY, South Bend, IN (US); Seth FAITH, Columbus, OH (US); Nick NASTASI, Columbus, OH (US); Nicole RENNINGER, Columbus, OH (US)

(21) Appl. No.: 18/290,623

(22) PCT Filed: Apr. 12, 2022
(86) PCT No.: PCT/US2022/024483

§ 371 Date: Jan. 19, 2024

Related U.S. Application Data
(60) Provisional application No. 63/224,667, filed on Jul. 22, 2021.

(51) Int. Cl. C12Q 1/70; C12Q 1/6806

(52) U.S. Cl. CPC C12Q 1/701; C12Q 1/6806; C12Q 2600/156

(57) ABSTRACT

Described herein are methods for the detection of a virus (e.g., SARS-CoV-2) RNA in dust, which can be used for continued environmental surveillance of the viral disease. Targeted monitoring of dust in high-concern buildings can complement broader population-level monitoring approaches. Additionally, a method for detection of a viral RNA in a dust sample is disclosed herein.

### CROSS REFERENCE TO RELATED APPLICATIONS

This application is a national stage application filed under 35 U.S.C. § 371 of PCT/US2022/024483, filed Apr. 12, 2022, which claims the benefit of U.S. Provisional Patent Application Ser. No. 63/224,667 filed Jul. 22, 2021, the disclosure of which is expressly incorporated herein by reference in its entirety.

### STATEMENT REGARDING FEDERALLY SPONSORED RESEARCH OR DEVELOPMENT

This invention was made with government support under grant/contract number R21 AI168817 awarded by the National Institutes of Health. The government has certain rights in the invention.

### REFERENCE TO SEQUENCE LISTING

Applicant submits herewith a Sequence Listing in computer readable form and in compliance with 37 C.F.R. § 1.821-1.825. This sequence listing is in ASCII TXT format with filename “103361-119US1_2024_08_30_Sequence Listing,” a 2,073 bytes file size, and creation date of Aug. 28, 2024. The content of the Sequence Listing is hereby incorporated by reference.

### BACKGROUND

COVID-19 has caused tens of millions of cases and millions of death since reaching pandemic designation in March 2020. The disease is spread by severe acute respiratory syndrome coronavirus 2 (SARS-CoV-2). Often, individuals are not symptomatic but may still spread the virus. Infected individuals spread the virus into the environment in respiratory droplets. Many droplets may land on the floor and other indoor surfaces.

We need new and improved methods to monitor for COVID-19 and other viral illnesses. We need techniques that could work at the building scale and complement individual testing and ongoing wastewater monitoring efforts. Wastewater samples can be difficult to collect and process, and not all individuals shed virus in feces. We need a more targeted approach to monitor for viruses in the indoor environment.

### SUMMARY

Described herein are methods for the detection of a virus (e.g., SARS-CoV-2) nucleic acid (e.g., viral RNA or viral DNA) in dust, which can be used for continued environmental surveillance of the viral disease (e.g., the novel coronavirus, SARS-CoV-2). Targeted monitoring of dust in high-concern buildings can complement broader population-level monitoring approaches. The examples described herein demonstrate that indoor dust can be used as a matrix for viral surveillance. Additionally, a method for detection of a viral nucleic acid (e.g., viral RNA or viral DNA) in a dust sample is disclosed herein.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

FIGS. 1A and 1B are heatmaps displaying the geometric mean number of copies per microliter of RNA template of samples above the detection limit (samples below detection limit were excluded) (FIG. 1A) and the proportion of samples positive (as a percentage) (FIG. 1B) for bulk dust, surface swabs, and passive sampler as observed with three PCR-based methods. The variable n is the number of samples used to calculate the geometric mean (positive detects) in panel A or proportion of samples positive in panel B. For “Floor Dust,” n refers to the total number of bulk dust samples tested for SARS-CoV-2, including the dust samples tested weekly over a period of 4 weeks. Items in white on the room diagram were not sampled, and items in white with a slash in the lower heatmaps were not detected. Colors on the room diagrams represent the average value among all three measurement methods.

FIG. 2A shows RNA concentration of bulk dust samples (average of four bags) from initial collection to 4 weeks as measured by ddPCR, dPCR, and qPCR. Error bars shown represent the 95% confidence interval of each measurement. FIGS. 2B-2D show cumulative normal probability plots for each measurement method show variability of RNA concentration values for each bulk bag collected.

FIG. 3 is an example computing device.

FIG. 4 is a graph illustrating environmental surveillance for a viral disease in enclosed structures of a campus. Different buildings are listed on the vertical axis and different weeks are listed on the horizontal axis. Different shades are used in the graph to represent different concentration levels (e.g., heatmap).

FIG. 5 is a graph illustrating variants of a viral disease sequenced from dust samples over time. The graph of FIG. 5 is a prophetic example provided for illustration only.

FIG. 6 shows size comparison among VOC, viruses, and droplets.

FIG. 7 shows that a new long-term surveillance solution is needed.

FIG. 8 shows methods of sample collection.

FIG. 9 shows that RNA detection is different than infectivity.

FIG. 10 shows detection works in residence hall.

FIG. 11 shows that RNA persists whereas viral viability decays rapidly.

FIG. 12 shows that dust can complement wastewater monitoring.

FIG. 13 shows indoor environmental quality. Elevated moisture is sufficient to support microbial growth and function in dust. Chemical emissions indoors are dominated by materials, but smells may come from microbes. Dust is an efficient matrix for COVID-19 surveillance.

### DETAILED DESCRIPTION

Unless defined otherwise, all technical and scientific terms used herein have the same meaning as commonly understood by one of ordinary skill in the art. Methods and materials similar or equivalent to those described herein can be used in the practice or testing of the present disclosure. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. The terms “optional” or “optionally” used herein mean that the subsequently described feature, event or circumstance may or may not occur, and that the description includes instances where said feature, event or circumstance occurs and instances where it does not. Ranges may be expressed herein as from “about” one particular value, and/or to “about” another particular value. When such a range is expressed, an aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. While implementations will be described for severe acute respiratory syndrome coronavirus 2 (SARS-CoV-2) monitoring in bulk floor dust and related samples, it will become evident to those skilled in the art that the implementations are not limited thereto, but are applicable for using indoor dust as a matrix for viral surveillance for other viruses of concern such as influenza A and B, respiratory syncytial virus (RSV), Rhinoviruses, Adenoviruses, and other emerging viral disease, such as Ebola/Marburg, Dengue, and Arenaviruses. Additional viruses may include but are not limited to Norwalk-like virus (norovirus), adenovirus, rhinovirus, other coronaviruses, parainfluenza, and others.

As used herein, the terms “about” or “approximately” when referring to a measurable value such as an amount, a percentage, and the like, is meant to encompass variations of ±20%, ±10%, ±5%, or ±1% from the measurable value.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

The term “primer” as used herein refers to an oligonucleotide, whether occurring naturally or produced synthetically, which is capable of acting as a point of initiation of synthesis when placed under conditions in which synthesis of primer extension product which is complementary to a nucleic acid strand (template) is induced, i.e., in the presence of nucleotides and an agent for polymerization such as DNA polymerase and at a suitable temperature and pH. The primer is preferably single stranded for maximum efficiency in amplification. Preferably, the primer is an oligodeoxyribonucleotide. The primer of this invention can be comprised of naturally occurring dNMP (i.e., dAMP, dGM, dCMP and dTMP), modified nucleotide or non-natural nucleotide. The primer can also include ribonucleotides. The primer must be sufficiently long to prime the synthesis of extension products in the presence of the agent for polymerization. The exact length of the primers will depend on many factors, including temperature, application and source of primer. The term “annealing” or “priming” as used herein refers to the apposition of an oligodeoxynucleotide or nucleic acid to a template nucleic acid, whereby said apposition enables the polymerase to polymerize nucleotides into a nucleic acid molecule which is complementary to the template nucleic acid or a portion thereof.

The term “nucleic acid” as used herein means a polymer composed of nucleotides, e.g., deoxyribonucleotides or ribonucleotides.

The terms “ribonucleic acid” and “RNA” as used herein mean a polymer composed of ribonucleotides.

The terms “deoxyribonucleic acid” and “DNA” as used herein mean a polymer composed 20 of deoxyribonucleotides.

The term “oligonucleotide” denotes single- or double-stranded nucleotide multimers of from about 2 to up to about 100 nucleotides in length. Suitable oligonucleotides may be prepared by the phosphoramidite method described by Beaucage and Carruthers, Tetrahedron Lett., 22:1859-1862 (1981), or by the triester method according to Matteucci, et al., J. Am. Chem. Soc., 103:3185 (1981), both incorporated herein by reference, or by other chemical methods using either a commercial automated oligonucleotide synthesizer or VLSIPS™ technology. When oligonucleotides are referred to as “double-stranded,” it is understood by those of skill in the art that a pair of oligonucleotides exist in a hydrogen-bonded, helical array typically associated with, for example, DNA. In addition to the 100% complementary form of double-stranded oligonucleotides, the term “double-stranded,” as used herein is also meant to refer to those forms which include such structural features as bulges and loops, described more fully in such biochemistry texts as Stryer, Biochemistry, Third Ed., (1988), incorporated herein by reference for all purposes.

The term “polynucleotide” refers to a single or double stranded polymer composed of nucleotide monomers.

“Polymerase chain reaction”, or “PCR”, generally refers to a method for amplification of a desired nucleotide sequence in vitro. Generally, the PCR process consists of introducing a molar excess of two or more extendable oligonucleotide primers to a reaction mixture comprising a sample having the desired target sequence(s), where the primers are complementary to opposite strands of the double stranded target sequence. The reaction mixture is subjected to a program of thermal cycling in the presence of a DNA polymerase, resulting in the amplification of the desired target sequence flanked by the DNA primers.

The term “dust” refers to the combination of particulate matter and debris that accumulates naturally in buildings on the floor and on surfaces over time. Dust is also present in the air.

Environmental surveillance to assess pathogen presence within a community is proving to be a critical tool to protect public health, and it is especially relevant during the ongoing COVID-19 pandemic. Importantly, environmental surveillance tools also allow for the detection of asymptomatic disease carriers and for routine monitoring of a large number of people as has been shown for SARS-CoV-2 wastewater monitoring. However, additional monitoring techniques are needed to screen for outbreaks in high-risk settings such as congregate care facilities. The examples described herein demonstrate that SARS-CoV-2 can be detected in bulk floor dust collected from rooms housing infected individuals. This analysis suggests that dust is a useful and efficient matrix for routine surveillance of viral disease.

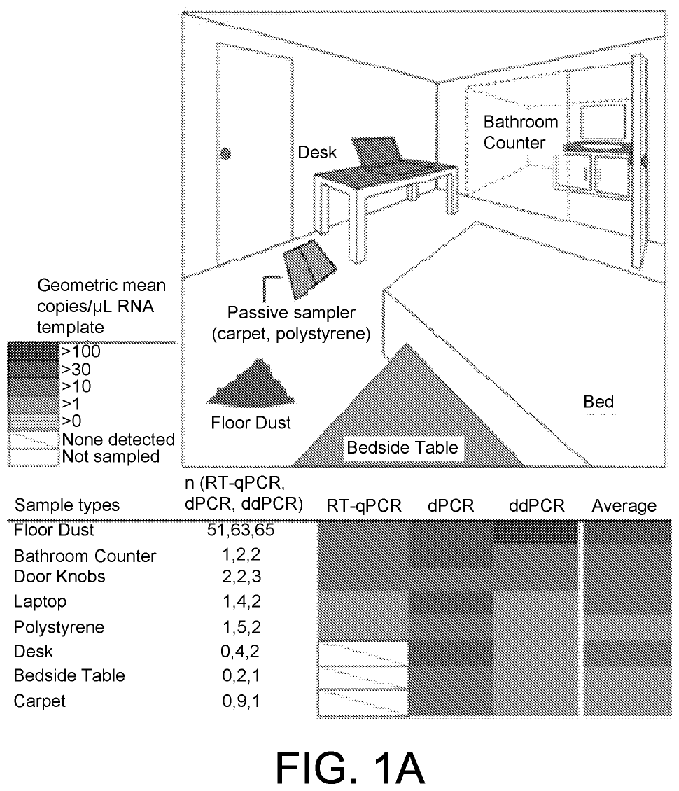

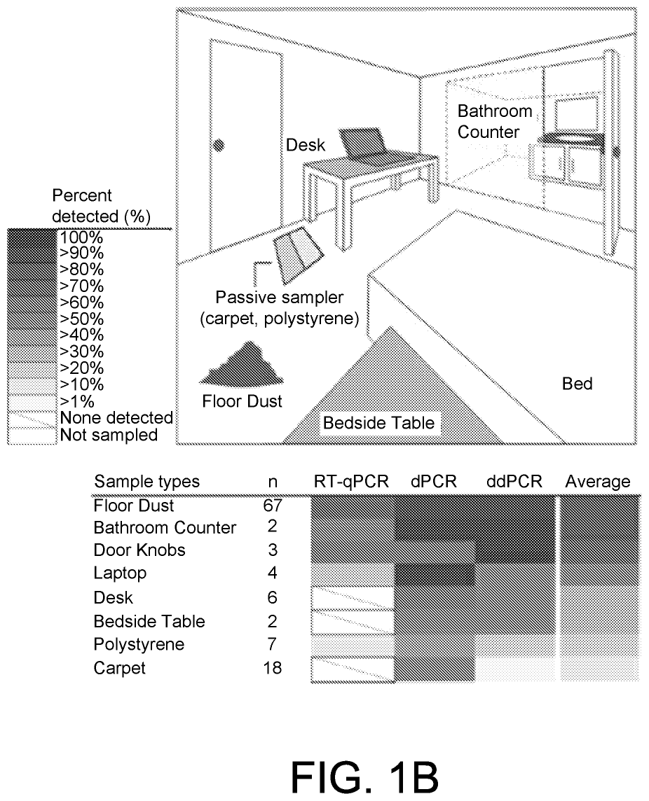

SARS-CoV-2 was measured using quantitative reverse transcription PCR (RT-qPCR), chip-based digital PCR (dPCR), and droplet digital PCR (ddPCR) in samples of bulk dust, passive surface samples, and surface swabs from rooms of individuals with COVID-19. In bulk dust, the SARS-CoV-2 viral concentration had a geometric mean value of 163 copies/mg of dust and ranged from nondetects to 23,049 copies/mg of dust (FIG. 1A). SARS-CoV-2 RNA was detected in 89% of bulk dust, 55% of surface swabs, and 21% of passive surface sampler samples (average among all three detection methods used, Kruskal-Wallis P=0.02). The ddPCR method detected viral RNA in 97% of bulk dust samples compared to 93% for the chip-based dPCR and 76% for RT-qPCR (Kruskal-Wallis P=0.37) (FIG. 1B). Across all sample types, the ddPCR method detected viral RNA in 60% of samples compared to 71% for the chip-based dPCR and 29% for RT-qPCR (Kruskal-Wallis P=0.06).

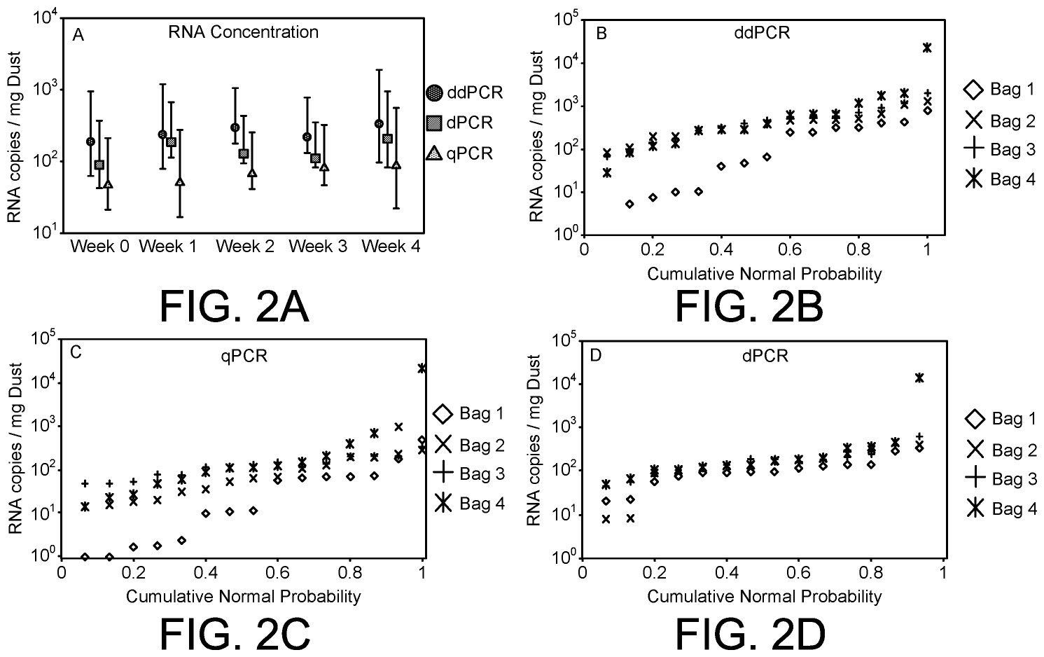

The COVID-19 isolation rooms were treated with a chlorine-based disinfectant prior to dust collection as part of the normal cleaning process, and the disinfectant is expected to largely inactivate the virus through reactions with the viral capsid (15). The bags were stored in the laboratory at room temperature after collection. Triplicate subsamples were extracted, and viral RNA was measured immediately upon collection and once per week for 4 weeks. Viral RNA did not measurably decay over 4 weeks in the vacuum bags (regression R²=0.009, P=0.47) (FIG. 2A). The coefficient of variance (CV) for number of copies/mg of dust ranged from 73.5% to 313.4% within each vacuum bag when averaged across the three methods of viral detection. This large variation in viral concentration is likely due to the heterogeneous mixture in the bags (FIG. 2B to 2D).

The novel coronavirus and the ongoing COVID-19 pandemic have highlighted the need for sensitive and scalable viral surveillance within communities. In the long term, the threat of COVID-19 outbreaks will subside to a level where indefinite routine testing of asymptomatic individuals may be too cumbersome or expensive. However, there will continue to be a need to more broadly monitor vulnerable populations such as those in long-term-care facilities or high-risk patients in hospitals for SARS-CoV-2, influenza, respiratory syncytial virus (RSV), and other emerging viral diseases. Novel pathogens can be targeted with adaptable PCR-based assays. After detection, outbreaks can then be addressed with more targeted resources such as direct patient testing.

The results herein demonstrate that environmental dust collection can provide a convenient and useful matrix for ongoing viral monitoring. The process can provide monitoring for many high-risk individuals, and dust samples are already being collected through normal cleaning practices such as vacuuming. Dust had a higher positivity rate than surface swab samples, and the positivity rate of the surface swabs in this study was similar to or greater than the rates in similar studies (16, 17). Our observations indicate that SARS-CoV-2 RNA in dust can persist at least 4 weeks after dust collection and that the measured concentration can vary in different dust subsamples within a vacuum bag. Therefore, multiple samples should be taken from a bag to more rigorously quantify the viral genetic signal, or homogenization methods should be developed that comply with biosafety standards. Additionally, RNA and dust persistence in the environment should be considered when determining if the outbreak occurred recently or in the past. Differences between PCR-based measurement methods may inform method choice. For instance, RT-qPCR requires calibration standards for quantification and the digital methods do not, and for the assays used, ddPCR is a one-step reaction and the chip-based dPCR requires a two-step reaction. Each instrument also has a different detection limit and resulted in marginally different positivity rates. Previously, measurements of indoor environmental microbes have been used to detect infectious microbes such as Aspergillus fumigatus and Legionella pneumophila (18-20). However, nucleotide-based tests do not measure infectivity, meaning the detection of genetic material from these microbes may indicate that people in the area are infected but would not necessarily indicate the risk of infection due to contact with indoor surfaces or via resuspension of floor dust.

Indoor dust can also be used to complement other environmental surveillance methods, e.g., wastewater monitoring. Wastewater detection may be more beneficial at larger population scales covering thousands of individuals in a community, and one infected individual can be detected among 100 to 2,000,000 individuals (21). Indoor dust can be useful in areas with smaller numbers of high-risk individuals where more specific outbreak identification is critical. Additionally, not all individuals secrete virus in stool (22). Indoor dust sampling can also be less expensive and be easier to implement, with simplified sample collection and no preconcentration steps of samples required. Other dust collection methods are available beyond those described in this study. Future research should evaluate differences between collection strategies.

Indoor dust provides an important matrix for environmental surveillance of viral disease outbreaks. Infected humans shed virus into their surrounding environment, which becomes integrated into the dust. In many cases, dust is already being collected during routine cleaning and can easily be submitted for analysis. Overall, dust can be a useful and efficient matrix to provide identification of viral disease in high-risk settings, such as congregate care facilities. Future research can validate these results on a broader scale and in different building types to better inform use of this technique to mitigate viral transmission.

### Example Surveillance Methods

An example method for environmental surveillance is described below. The method includes collecting a dust sample from an area inside an enclosed structure. This disclosure contemplates that the enclosed structure is a building such as a house, hotel/motel, school, office, medical facility, etc. As described herein, the dust sample is a bulk dust sample in some implementations. In other implementations, the dust sample is a surface swab sample or a passive surface sample. Optionally, the method includes sieving the dust sample (e.g., to 250 μm or 300 μm in diameter or any other size). The method also includes extracting viral RNA from the dust sample. Techniques for extracting viral RNA from the dust sample are described below. It should be understood that many viruses will likely not be viable when measured (especially for enveloped viruses). See Nicholas Nastasi et al., “Viability of MS2 and Phi6 Bacteriophages on Carpet and Dust,” doi.org/10.1101/2021.05.17.444479. This offers a distinct advantage because 1) the samples do not need to be stored cold and can be used at least out to 4 weeks after collection and 2) for enveloped viruses especially this reduces the biosafety hazard of handling the dust. Additionally, the method includes quantifying an amount of the extracted viral RNA contained in the dust sample. For example, this can be accomplished by performing a PCR assay on the extracted viral RNA. As described herein, the PCR assay is a RT-qPCR assay in some implementations. In other implementations, the PCR assay is a digital PCR assay (e.g., dPCR or ddPCR assay). Example PCR assays are described below. It should be understood that PCR-based detection is provided only as an example. This disclosure contemplates using other techniques to quantify the amount of the extracted viral RNA contained in the dust sample. This may include using a lateral flow test or loop-mediated amplification (LAMP) technology, for example. Lateral flow assay is also described in the art, including U.S. Pat. No. 8,399,261; U.S. Publication No. 20150293086A1; each of which is herein incorporated by reference.

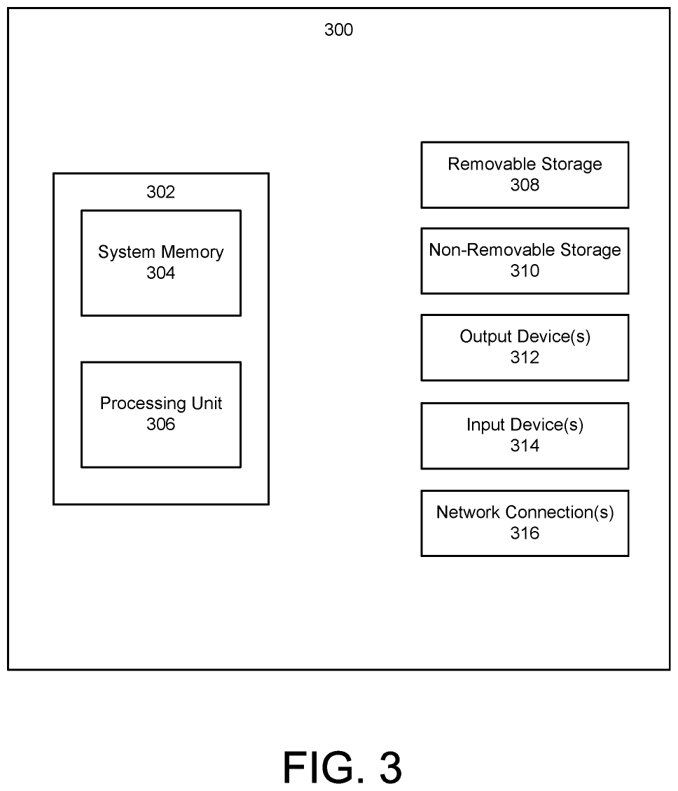

The method further includes determining whether a viral disease (e.g., SARS-CoV-2) is present inside the enclosed structure based on the amount of the extracted viral RNA contained in the dust sample. This disclosure contemplates that the determination can be performed by a computing device (e.g., computing device shown in FIG. 3). For example, the amount of the extracted viral RNA contained in the dust sample can be received at a computing device and then compared to a threshold value. If the amount of the extracted viral RNA contained in the dust sample exceeds the threshold value, then the viral disease is present inside the enclosed structure. Otherwise, the viral disease is not present inside the enclosed structure. In some implementations, there are a plurality of threshold values, each representing a different level of risk. For example, samples<1 copies/mg dust are “green,” samples 1-<10 copies/mg dust are “yellow,” and samples>=10 copies/mg dust are “red”. It should be understood that <1, 1-<10, >=10 are threshold values.

In one example implementation where the viral disease is severe acute respiratory syndrome coronavirus 2 (SARS-CoV-2) including delta and omicron variants thereof, the threshold value can be between about 50 copies/mg dust and about 1000 copies/mg dust. For example, the threshold value may be about 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 copies/mg dust. Optionally, the threshold value can be between about 200 copies/mg dust and about 400 copies/mg dust. For example, the threshold value may be about 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, or 400 copies/mg dust. Optionally, the threshold value can be between about 300 copies/mg dust. For example, the threshold value may be about 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, or 325 copies/mg dust. In some embodiments, SARS-CoV-2 is an alpha variant (e.g., B.1.1.7 or Q lineages), a beta variant (e.g., B.1.351 or descendent lineages), a gamma variant (e.g., P.1 and descendent lineages), an epsilon variant (e.g., B.1.427 or B.1.429), an eta variant (e.g., B.1.525), an iota variant (e.g., B.1.526), a kappa (e.g., B.1.617.1), 1.617.3, a mu variant (e.g., B.1.621, B.1.621.1), a Zeta variant (e.g., P.2), a Delta variant (e.g., B.1.617.2 or AY lineages), or an omicron variant (e.g., B.1.1.529 or BA lineages). In some examples, disclosed herein is a method of detection and/or monitoring any variants of SARS-CoV-2.

In some embodiments, the method disclosed herein further comprises disinfecting the tested area and/or the dust sample if the amount of the detected viral nucleic acid in the dust sample is above the threshold value. In some embodiments, the threshold value is between about 50 copies per milligram (mg) dust and about 1000 copies per mg dust. In some embodiments, the threshold value is about 300 copies per mg dust.

It should be understood that the threshold value will vary with the type of viral disease. In some implementations, the threshold value may be the detection threshold (i.e., detected/not detected), for example, for a deadly disease such as the Ebola virus. While in other implementations, the threshold value is greater than the detection threshold and also specific to the viral disease. Thus, the threshold value for viral diseases other than SARS-CoV-2 may be different than the example threshold values provided above.

Optionally, an action item from a risk management plan can be recommended based on the comparison. This disclosure contemplates that the recommendation can be provided by a computing device (e.g., computing device shown in FIG. 3). For example, the action item is to test individuals for the viral disease. Alternatively or additionally, the action item is to take a personal protective measure and/or use personal protective equipment. Alternatively or additionally, the action item is to sequence the extracted viral RNA to detect variants. It should be understood that the individual testing and sequencing are provided only as example action items. This disclosure contemplates that the risk management plan can include more, less, and/or different action items than the examples. For example, action items may include, but are not limited to, mandated masking, social distancing, building lockdown, enhanced cleaning, and improved ventilation.

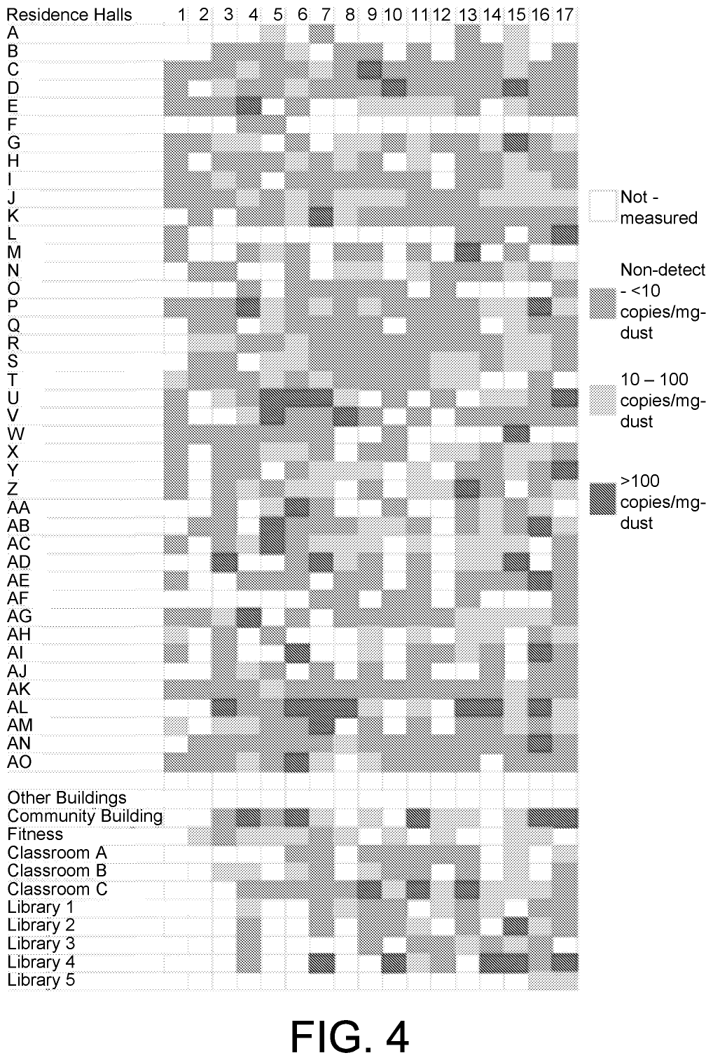

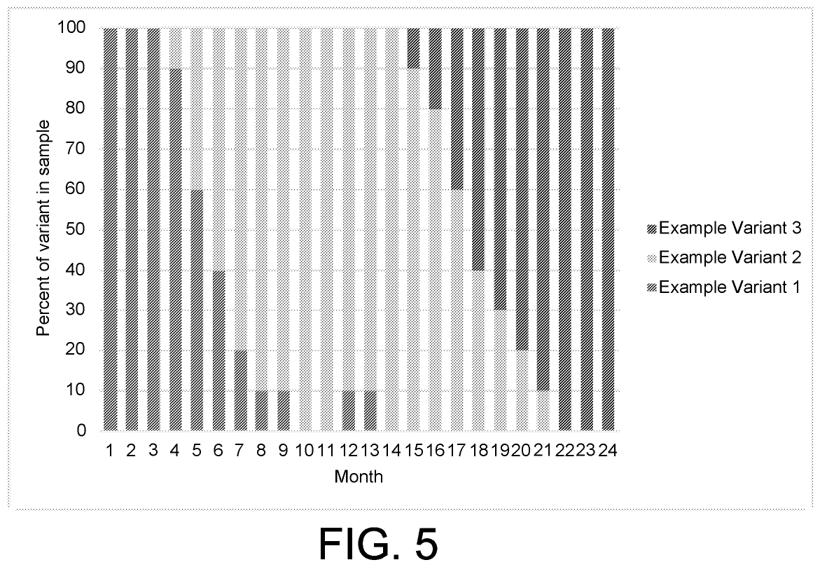

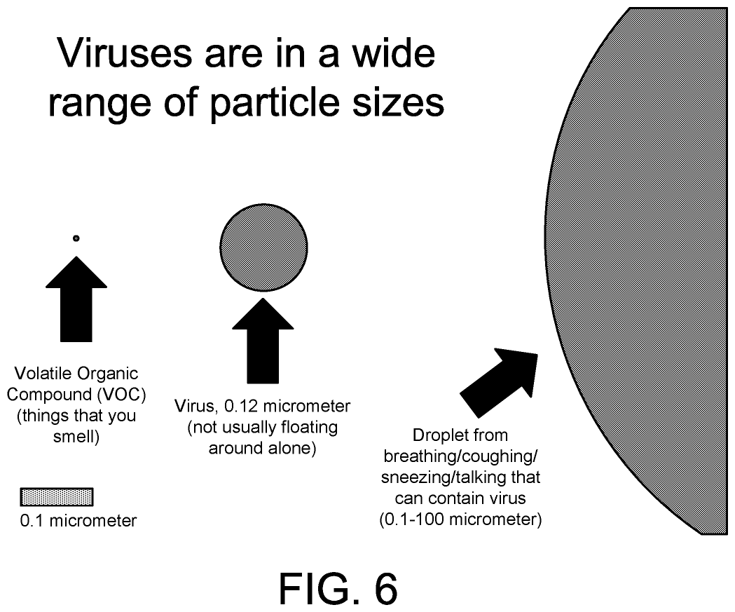

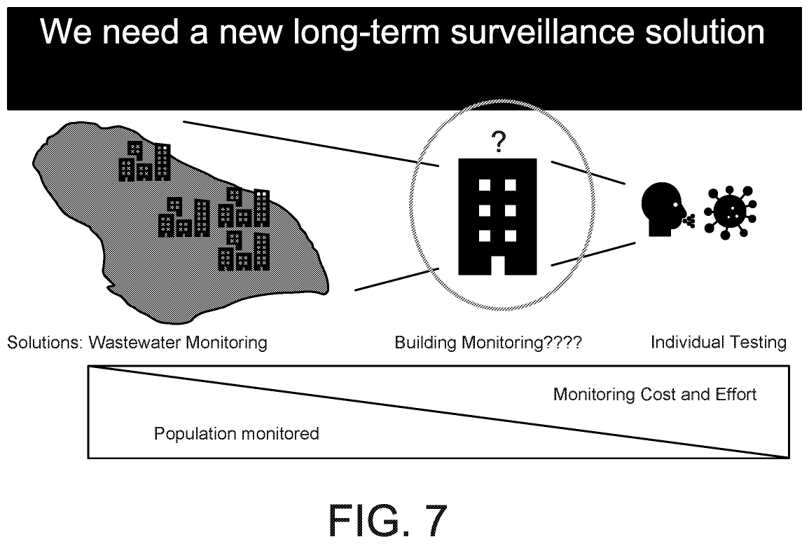

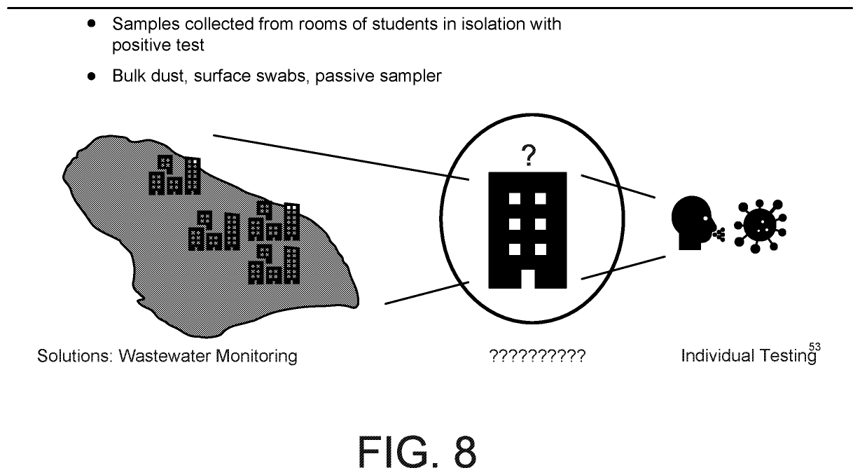

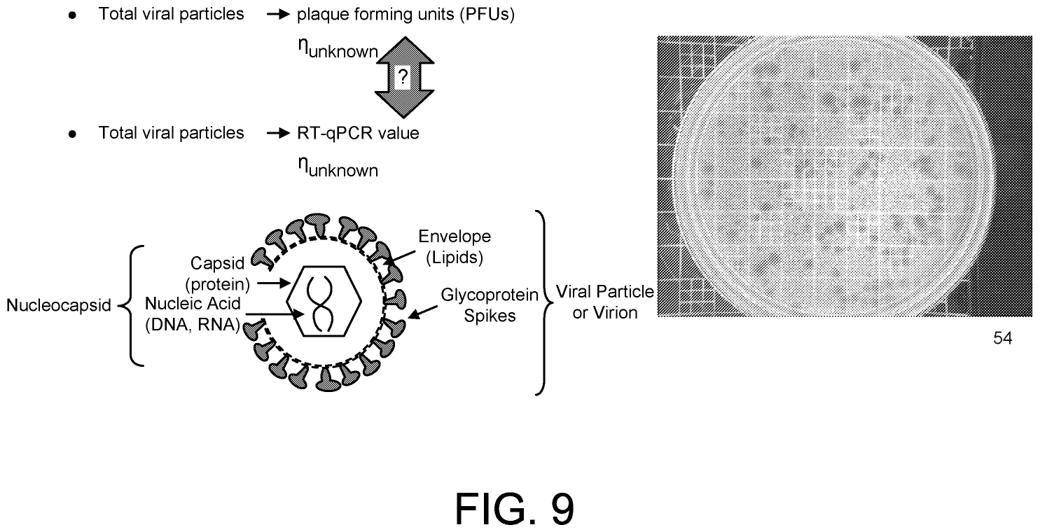

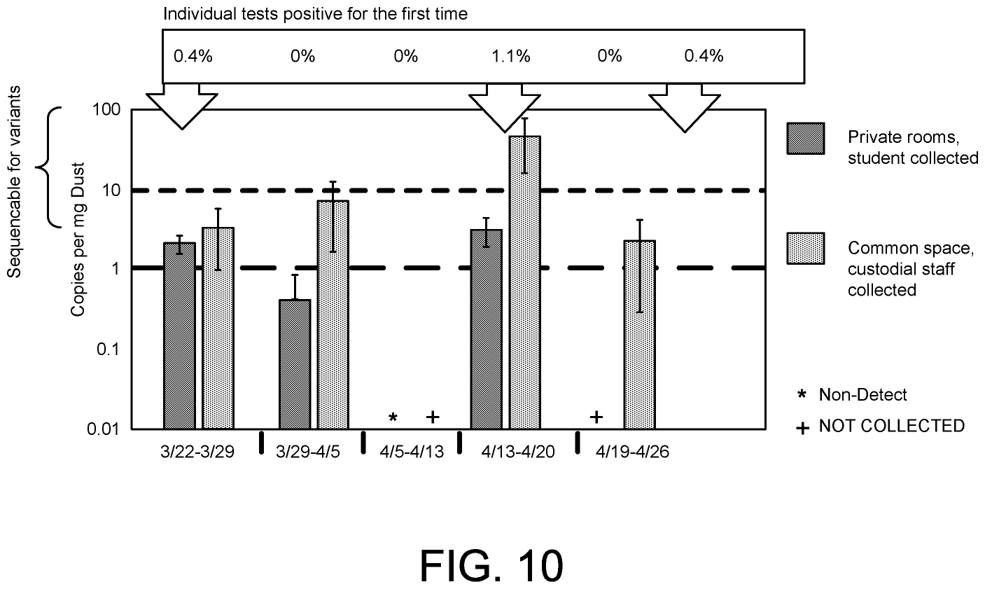

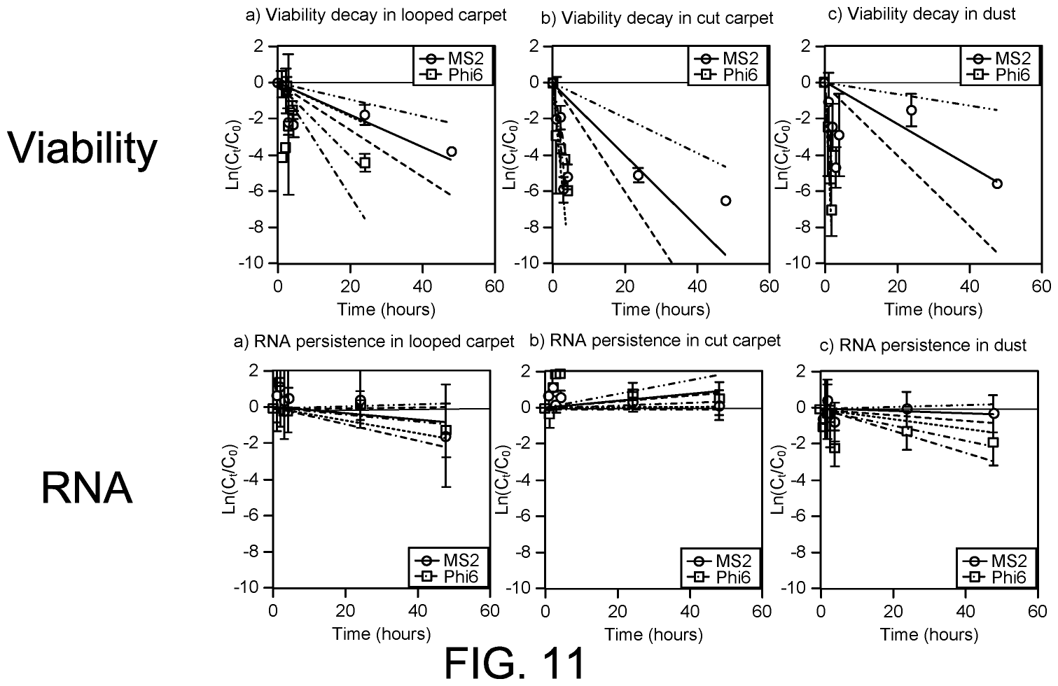

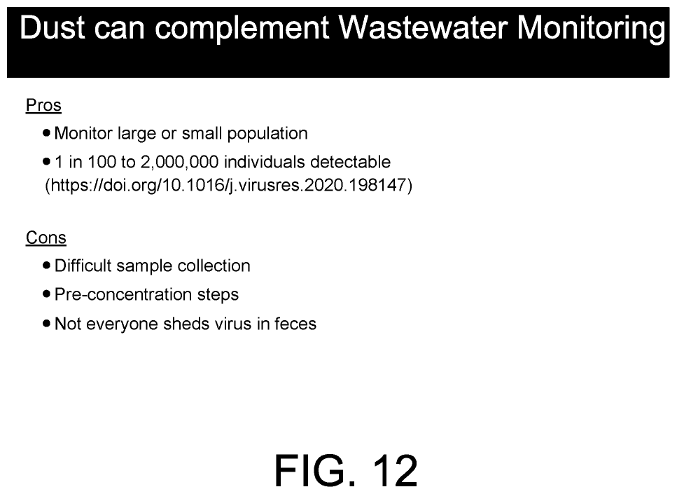

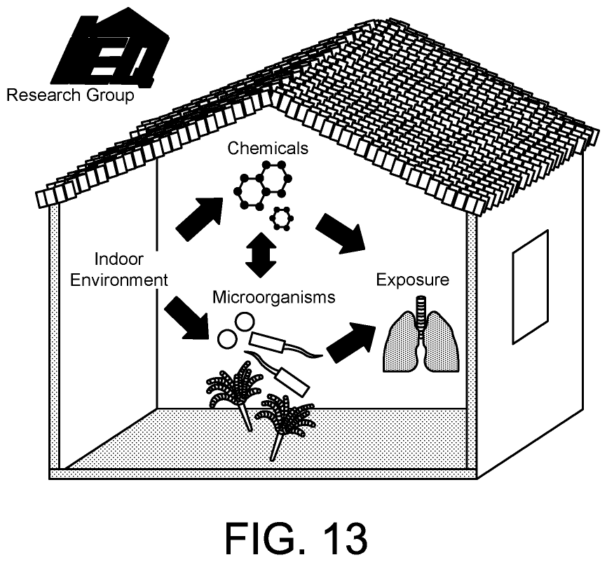

Optionally, a report including the amount of the extracted viral RNA contained in the dust sample can be generated. Heatmaps such as those shown in FIGS. 1A and 1B and RNA concentration and cumulative norm probability plots shown in FIGS. 2A-2D are example reports. Alternatively or additionally, reports similar to those shown by FIG. 4 (heatmap for environmental surveillance of structures over time) and FIG. 5 (percentage of variants in dust samples) can be provided. This disclosure contemplates that the report can be generated by a computing device (e.g., computing device shown in FIG. 3). It should be understood that FIGS. 1A-2D are provided only as example reports. This disclosure contemplates that the reports can be other than those shown in the figures. For example, a report may include color-coded buildings on a map, where different colors indicate respective viral concentrations within the buildings. Optionally, an action item from a risk management plan can be recommended based on the report. For example, this disclosure contemplates that reports similar to those shown by FIG. 4 and/or FIG. 5 can be used to understand the spread of the viral disease by location and variant over time and such information may be used to inform actions.

Optionally, the amount of the extracted viral RNA contained in the dust sample can be correlated to an approximate number of infected individuals using the enclosed structure. This disclosure contemplates that the correlation can be performed by a computing device (e.g., computing device shown in FIG. 3).

Optionally, the method further includes sequencing viral RNA that has been extracted from the dust sample to detect variants. Techniques for sequencing viral RNA are described herein.

### Example Methods for Detection

Disclosed herein is a method for detection of a viral nucleic acid in a dust sample, comprising
a) extracting the viral nucleic acid from the dust sample;
b) determining a level of inhibition in the viral nucleic acid extracted from the dust sample;
c) diluting the viral nucleic acid extracted from the sample if the level of inhibition is increased in comparison to a control; and
d) quantifying an amount of the diluted viral nucleic acid.

In some embodiments, the viral nucleic acid is a viral RNA or viral DNA. Methods for nucleic acid extraction are known in the art. See, e.g., U.S. Pat. Nos. 9,738,931 and 9,580,751, incorporated by reference herein in their entireties. In some examples, step a) comprises phenol-based lysis.

Step a) of the method disclosed herein comprises using an RNase inhibitor, wherein the RNase inhibitor can be any composition known in the art that can inactivate, denature, and/or inhibit a ribonuclease. Beta-mercaptoethanol (BME) is a reducing agent that can irreversibly denature RNases. In some examples, step a) of the method disclosed herein comprises using a concentration of beta-mercaptoethanol at least about 1.5 times greater (for example, at least about 1.5 times greater, at least about 2 times greater, at least about 3 times greater, at least about 4 times greater, at least about 5 times greater, at least about 6 times greater, at least about 7 times greater, at least about 8 times greater, at least about 9 times greater, at least 10 times greater, at least about 20 times greater, at least about 40 times greater, at least about 50 times greater, or at least 100 times greater) than a recommended concentration. In some embodiments, step a) comprises using a concentration of beta-mercaptoethanol about 10 times greater than a recommended concentration. Dust contains high concentrations of RNases that must be denatured with beta-mercaptoethanol or other protein denaturing compounds prior to extraction. The recommended concentration of beta-mercaptoethanol can be, for example, about 1%, 2%, 5%, 10%, or 20% of a lysis buffer. In some embodiments, the concentration of beta-mercaptoethanol used in the method disclosed herein is about 10%, 20%, or 50% of a lysis buffer (v/v). In some embodiments, the concentration of beta-mercaptoethanol used in the method disclosed herein is about 8% to about 12% or about 9% to about 11% of a lysis buffer (v/v). In some embodiments, the concentration of beta-mercaptoethanol used in the method disclosed herein is about 10% of a lysis buffer (v/v). In some embodiments, an alternative compound that denatures proteins and specifically RNases can be used.

In some examples, step b) of the method disclosed herein comprises
adding a nucleic acid template into the viral nucleic acid (e.g., viral RNA or viral DNA) extracted from the dust sample;
performing a PCR assay to amplify the nucleic acid template; and
determining that the level of inhibition is increased if the amplification of the nucleic acid template is delayed in comparison to a reference control.

In some examples, the level of inhibition is increased if the amplification of the nucleic acid template is delayed by at least about 1 cycle (for example, at least about 1.2 cycles, at least about 1.4 cycles, at least about 1.6 cycles, at least about 1.8 cycles, at least about 2 cycles, at least about 4 cycles, at least about 10 cycles, at least about 50 cycles, or at least about 100 cycles). The term “reference control” refers to a level detected in a sample without the addition of the viral nucleic acid (e.g., viral RNA or viral DNA) extracted from the dust sample.

The technique of PCR is described in numerous publications, including, PCR: A Practical Approach, M. J. McPherson, et al., IRL Press (1991), PCR Protocols: A Guide to Methods and Applications, by Innis, et al., Academic Press (1990), and PCR Technology: Principals and Applications for DNA Amplification, H. A. Erlich, Stockton Press (1989). PCR is also described in many U.S. patents, including U.S. Pat. Nos. 4,683,195; 4,683,202; 4,800,159; 4,965,188; 4,889,818; 5,075,216; 5,079,352; 5,104,792; 5,023,171; 5,091,310; and 5,066,584, each of which is herein incorporated by reference. In some examples, the PCR assay is a quantitative reverse transcription PCR (RT-qPCR) assay or a digital PCR assay (for example, a chip-based digital PCR (dPCR) or droplet digital PCR (ddPCR) assay).

In some examples, the method disclosed herein further comprises sequencing the viral nucleic acid (e.g., viral RNA or viral DNA) extracted from the dust sample. The sequencing techniques are known in the art, including, for example, Maxam-Gilbert sequencing, chain-termination methods, shotgun sequencing, single molecule real time (SMRT) sequencing, nanopore DNA sequencing, short-read sequencing methods, massively parallel signature sequencing (MPSS), polony sequencing, 454 pyrosequencing, illumina (Solexa) sequencing, combinatorial probe anchor synthesis (cPAS), SOLID sequencing, lon Torrent semiconductor sequencing, DNA nanoball sequencing, or heliscope single molecule sequencing. In some examples, the viral nucleic acid (e.g., viral RNA or viral DNA) is reverse transcribed and amplified by PCR before the step of sequencing.

In some examples, the viral RNA or DNA with a virus selected from the group consisting of Herpes Simplex virus-1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus (including, but not limited to avian coronavirus (IBV), porcine coronavirus HKU15 (PorCoV HKU15), Porcine epidemic diarrhea virus (PEDV), HCoV-229E, HCoV-OC43, HCoV-HKU1, HCoV-NL63, SARS-CoV, SARS-CoV-2, or MERS-CoV), Influenza virus A, Influenza virus B, Measles virus, Polyomavirus, Human Papillomavirus, Respiratory syncytial virus, Adenovirus, Coxsackie virus, Dengue virus, Mumps virus, Poliovirus, Rabies virus, Rous sarcoma virus, Reovirus, Yellow fever virus, Zika virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, and Human Immunodeficiency virus type-2. In some embodiments, the viral RNA is a SARS-CoV-2 RNA.

In some examples, the method disclosed herein comprises the steps in the process:
Vacuum dust.
Remove dust from bag and sieve to 250 μm or 300 μm in diameter or other sieve sizes. This must be done in a biosafety cabinet and benefits from the use of mechanical sieving machines.
Extract dust
Dilute dust and measure inhibition.
Run RT-qPCR.
If desired and sample is positive and at high enough concentration (˜2.5 copies/mg dust), sequence for variants.
Report results
   Samples<1 copies/mg dust are “green,” samples 1-<10 copies/mg dust are “yellow,” and samples>=10 copies/mg dust are “red”
   These results are integrated into a risk management plan combined with wastewater and individual level testing. More individual testing for a building is triggered at either “yellow” or “red” levels depending on the risk tolerance for the building occupants.
   Detected variants can be used in the decision process. For instances, some variants may be more highly transmission, cause worse outcomes, or cause disease in vaccinated individuals.

The commercial kit used for the extraction step can be the Qiagen RNeasy Powermicrobiome extraction kit or other extraction methods can be used. The following modifications are made:
Use 10 times the recommended amount of betamercaptoethanol than recommended in the kit (e.g., adding 65 μl to 650 μl PM1 solution when using the RNeasy® PowerMicrobiome® Kit, or Promega Maxwell® RSC simplyRNA Tissue Kit).
Use phenol-chloroform-isoamyl alcoholDependent upon inhibition test, may run samples at different dilutions in qPCR. Samples are diluted with molecular water (laboratory grade water)
   Dilutions to include are 1× (not diluted) to 10,000× (though you would generally never go past 100-1000× so the 10,000× is just to be inclusive)
Test samples for inhibition. Dust contains many inhibitors (both known and unknown) that can interfere in the reaction chemistry for PCR-based reactions.
   Replace some of the water in the mastermix recipe with RNA template that is at least 10-100 times greater than the concentration in the highest sample.
   Distribute mastermix to wells on plate
   Add sample to each well (samples are run as single wells). Run 7 wells with no sample (add water or final elution buffer from extraction)
   Compare each sample to the 7 “blank” wells. If they are statistically lower, then that sample may have inhibition and needs to be run at a different dilution. That new dilution also needs to be tested for inhibition.
   For difficult samples that don't have a “sweet spot,” you can try adding bovine serum albumin (BSA)

For sequencing, the extraction described above is critical to have the highest-quality template. Samples can be amplified in triplicate and the products pooled prior to sequencing to provide higher rates of success. Trying different dilutions and adding BSA can also be helpful.

Sars-CoV-2 Sequencing from Dust
Extracted RNA (50 ng-200 ng) are reverse transcribed and amplified by polymerase chain reaction (PCR) with the ARTIC SARS-CoV-2 FS Library Prep Kit (New England Biolabs, Ipswich MA).
Amplified products are converted into Illumina sequencing libraries using the RNA Prep with Enrichment (L) Tagmentation Kit protocol (Illumina, San Diego, CA, USA) with unique dual indexes allowing for up to 384 sample analysis.
Barcoded/indexed sequencing libraries are pooled and quantified using ProNex NGS Library Quant Kit (NG1201, Promega Co. Madison, WI).
^{˜}650 pM libraries are loaded on P3 or P2 sequencing cartridges and analyzed with the NextSeq2000 (Illumina) with 2×100 bp cycles.
Data are transmitted to the BaseSpace Cloud platform (Illumina) and converted to FASTQ file format using DRAGEN (Illumina).
DRAGEN COVID Lineage app v3,5,1 (Illumina) is used to align sequence data and produce quality metrics, variant calls, consensus genome sequences, and variant tables.
Custom scripts (R programming language) are used to deconvolute mixed samples (more than one virus) to determine the probability of specific SARS-CoV-2 lineages.

### Example Computing Device

It should be appreciated that the logical operations described herein with respect to the various figures may be implemented (1) as a sequence of computer implemented acts or program modules (i.e., software) running on a computing device (e.g., the computing device described in FIG. 3), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

Referring to FIG. 3, an example computing device 300 upon which the methods described herein may be implemented is illustrated. It should be understood that the example computing device 300 is only one example of a suitable computing environment upon which the methods described herein may be implemented. Optionally, the computing device 300 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, computing device 300 typically includes at least one processing unit 306 and system memory 304. Depending on the exact configuration and type of computing device, system memory 304 may be volatile (such as random access memory (RAM)), non-volatile (such as read-only memory (ROM), flash memory, etc.), or some combination of the two. This most basic configuration is illustrated in FIG. 3 by dashed line 302. The processing unit 306 may be a standard programmable processor that performs arithmetic and logic operations necessary for operation of the computing device 300. The computing device 300 may also include a bus or other communication mechanism for communicating information among various components of the computing device 300.

Computing device 300 may have additional features/functionality. For example, computing device 300 may include additional storage such as removable storage 308 and non-removable storage 310 including, but not limited to, magnetic or optical disks or tapes. Computing device 300 may also contain network connection(s) 316 that allow the device to communicate with other devices. Computing device 300 may also have input device(s) 314 such as a keyboard, mouse, touch screen, etc. Output device(s) 312 such as a display, speakers, printer, etc. may also be included. The additional devices may be connected to the bus in order to facilitate communication of data among the components of the computing device 300. All these devices are well known in the art and need not be discussed at length here.

The processing unit 306 may be configured to execute program code encoded in tangible, computer-readable media. Tangible, computer-readable media refers to any media that is capable of providing data that causes the computing device 300 (i.e., a machine) to operate in a particular fashion. Various computer-readable media may be utilized to provide instructions to the processing unit 306 for execution. Example tangible, computer-readable media may include, but is not limited to, volatile media, non-volatile media, removable media and non-removable media implemented in any method or technology for storage of information such as computer readable instructions, data structures, program modules or other data. System memory 304, removable storage 308, and non-removable storage 310 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 306 may execute program code stored in the system memory 304. For example, the bus may carry data to the system memory 304, from which the processing unit 306 receives and executes instructions. The data received by the system memory 304 may optionally be stored on the removable storage 308 or the non-removable storage 310 before or after execution by the processing unit 306.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

### Experimental Protocol

Overview. Samples were collected from two different homes, as well as isolation rooms used to quarantine individuals who tested positive for SARS-CoV-2. Bulk dust was collected from both homes, and four composite samples were collected from 30 to 50 student isolation rooms each. Surface swabs and a passive sampler collection were completed in one home. Viral RNA was measured using RT-qPCR, chip-based digital PCR, and droplet digital PCR.

House surface swabs and passive sampler collection. Surface and passive samples were collected at the end of the 10-day quarantine period from two bedrooms of individuals who tested positive for SARS-CoV-2 in house 1. Surfaces were swabbed using sterile flocked swabs (Puritan, ME, USA), and passive samplers consisting of carpet coupons and polystyrene coupons were placed on the floors of their isolation rooms. The passive samplers consisted of three cut pile carpet squares (fiber length, 10 mm), three loop pile carpet squares (fiber length, 7.5 mm), and three polystyrene squares attached to a template. All squares were 5 cm×5 cm each. Both carpet types used were made of 100% polyethylene terephthalate (PET) fibers and a synthetic jute backing material. Fibers were specifically manufactured to contain no antimicrobial, stain, or soil resistance coatings. Swabs were dipped in autoclaved phosphate-buffered saline (PBS), and each was used on a different 10 cm×10 cm surface area. Each wetted swab was wiped left and right across the 10 cm×10 cm surface area, rotated ⅓ turn, and wiped to cover the surface area up and down, rotated a final ⅓ turn, and wiped in circular motions across the surface area. Swabs were placed back in the corresponding tube and resealed until extraction.

In room 1, two swabs were used for the desk, two for a bedside table, and two for a computer. A passive sampler was placed on the floor by the bed for 4 days. In room 2, two swabs were used for 100-cm² areas on a computer, two for the same areas on a desk, two for the same areas on a second desk, one for the doorknob of the bedroom, two for 100-cm² areas on the bathroom counter, and two on the bathroom doorknob, one for each side of the door. A passive sampler was placed on the floor between the desk and bed for 2 days, and another was placed on the open space in the bedroom for 4 days.

House bulk dust. Bulk floor dust was collected from occupant vacuum bags of two different houses that had individuals infected with COVID-19. House 1 had floor dust collected 27 days after quarantine ended. House 2 had floor dust collected in the middle of the quarantine period.

Isolation room bulk dust. Bulk dust samples were retrieved from four different vacuum bags used to clean the isolation rooms for students with COVID-19 at Ohio State University in Columbus, OH, USA, and extracted over 4 weeks. Vacuum bags were collected by cleaning staff from rooms that were used to house students who tested positive for SARS-CoV-2. One or two students would isolate in the rooms for 10 days after a positive diagnosis. The cleaning staff would vacuum and clean the rooms after the quarantine period was over and within 18 h of the students leaving the rooms. Cleaning staff would spray the room with an electrostatic sprayer containing a disinfectant (sodium dichloro-s-triazinetrione, CAS 2893-78-9) and wait at least 20 min prior to vacuuming. This disinfectant provides free chlorine (stabilized by cyanuric acid), which nonselectively oxidizes biomolecules to inactivate pathogens. It is possible the disinfectant may be depleted by reacting with other organic material and biomolecules (dead skin, etc.) and viral capsids in dust samples before impacting viral RNA (15). Cleaning staff used a Windsor Sensor XP12 vacuum (Kärcher, Denver, CO) to collect dust over a 3- to 4-week period. Each vacuum bag contained dust from approximately 30 to 50 isolation rooms as well as hallways, and potentially from surfaces in the isolation rooms if considered dusty. The isolation room flooring was vinyl composite tile, and the hallway flooring was wall to wall carpet.

RNA extraction. Viral RNA was extracted from dust and surface samples. Bulk dust samples and surface swabs were extracted using a Qiagen RNeasy Powermicrobiome extraction kit procedure (Qiagen, Hilden, Germany) modified to include 10 times the recommended concentration of 2-mercaptoethanol and using phenol-based lysis. Triplicates of approximately 50 mg of dust were removed from bulk dust samples using an autoclaved spatula, and each replicate was extracted individually in a laminar flow biosafety cabinet. The spatula was flame sterilized between removing replicates. Isolation room bulk dust was extracted over a period of 4 weeks in which triplicate dust samples were extracted from the same bag after initial collection and then again each week for 4 weeks for a total of 60 samples of this type. Bulk dust from student isolation rooms was stored in sealed bags and kept at a room temperature of approximately 22.8° C. with a room relative humidity that fluctuated from 15 to 30%. Dust was not sieved due to biosafety concerns. Swabs were placed directly into the lysis tubes for extraction. Carpet samples from the passive sampler were extracted using the QIAmp DSP Viral RNA minikit (Qiagen, Hilden, Germany). A 3 cm×1 cm area was cut out of the middle of each carpet to reduce potential edge effects and vortexed for 1 min in 4,000 μl of autoclaved PBS. A total of 140 μl of this wash liquid was used in the RNA extraction. Swabs were dipped in autoclaved PBS and wiped horizontally and vertically across the polystyrene pieces on the passive sampler. All extraction sets included a blank to detect potential contamination. The RNA extract of a prepandemic dust sample collected in September 2019 was also tested and shown to be negative for SARS-CoV-2 on RT-qPCR with no amplification.

Viral Detection RT-qPCR. The viral detection assay targeted the N1 gene using the IDT SARS-CoV-2 (2019-nCOV) CDC qPCR probe assay (Integrated DNA Technologies, Inc., Coralville, IA, USA). This assay uses the 2019-nCOV_N1 forward primer (GAC CCC AAA ATC AGC GAA AT—SEQ ID NO: 1), the 2019-nCov_N1 reverse primer (TCT GGT TAC TGC CAG TTG AAT CTG—SEQ ID NO: 2), and the 2019-nCOV_N1 probe (6-carboxyfluorescein [FAM]-ACC CCG CAT/ZEN/TAC GTT TGG ACC-3′ Iowa Black FQ [3IABkFQ]—SEQ ID NO: 3). Direct one-step real-time qPCR amplification of cDNA was performed using qScript XLT one-step RT-qPCR ToughMix (Quanta BioSciences, Gaithersburg, MD, USA). Each well contained 5 μl of RNA template, 10 μl of qScript XLT one-step RT-qPCR ToughMix, 1.5 μl of the IDT SARS-CoV-2 forward and reverse primers at 500 nM and probe at 125 nM, and 3.5 μl of sterile deionized (DI) water. The 2019-nCOV plasmid control 10-fold serial dilutions were used as a standard curve to calculate the number of copies per microliter of RNA template, based on plasmid quantification determined by dPCR (see below) (Integrated DNA Technologies, Inc., Coralville, IA, USA). Cycling parameters were set following the instructions supplied by the CDC for qScript XLT one-step RT-qPCR ToughMix (23). Cycling parameters consisted of 10 min at 50° C. for 1 cycle, 3 min at 95° C. for 1 cycle, and 3 s at 95° C. followed by 30 s at 55° C. for 50 cycles. Seven no-template controls were tested, and no amplification occurred.

A subset of 10% of samples were tested for inhibition. The RNA template was spiked with positive plasmid control to test for a reduction in signal due to the presence of inhibitors. The spike concentration was 100 times the highest sample concentration determined by qPCR. Inhibition was indicated if there was a delay in expected amplification. Each sample type was tested for inhibition: bulk dust, swab, and passive sampler. No inhibition was detected in any of the sample types except for the carpet wash from the passive sampler, where the inhibition delayed amplification by 1.45 cycles. Diluting these samples by 10-fold to reduce inhibition would place these samples below the detection limit of 2.3 copies per μl of RNA.

Viral Detection Chip-based dPCR. Digital PCR was performed using the QuantStudio 3D Digital (QS3D) PCR system (Applied Biosystems, Forest City, CA) that utilizes a chip-based technology. This system uses a QuantStudio 3D Digital PCR Chip Adapter kit for the ProFlex Flat Block thermal cycler equipped with a tilt base, which holds the chips (version 2) in place during thermocycling. cDNA was first reverse transcribed from RNA samples using the iScript cDNA Synthesis kit (Bio-Rad, Hercules, CA) according to the recommended reaction protocol on the ProFlex PCR system (Applied Biosystems, Forest City, CA). RNA was detected and quantified using the N1 assay described above. Each reaction was prepared as a 15-μl volume consisting of 2.00 μl of water, 7.25 μl of QuantStudio 3D Digital PCR Master Mix v2 (Applied Biosystems, Forest City, CA), forward and reverse primers at 500 nM, probe at 125 nM, and 5 μl of RNA extract. A portion (14.5 μl) of the solution was transferred into the sample loading port of the loading blade and then loaded onto the chip. Immersion fluid was used to cover the surface of the chip. The chip was then sealed, and additional immersion fluid was added to fill the chip case. Thermal cycling consisted of 10 min at 96° C., 39 cycles of 60° C. for 2 min followed by 98° C. for 30 s, and finally 60° C. for 2 min. The cover temperature was set at 70° C., and the reaction volume was set at 1 nl. Each experiment included one negative control and one N1 positive control (2019-nCOV_N_Positive Control; Integrated DNA Technologies, Inc., Coralville, IA, USA). Chips were then removed and imaged using the QuantStudio 3D digital PCR instrument following thermal cycling. Manual thresholding and quantification were performed using the QuantStudio 3D AnalysisSuit Software. The 95% limit of blank for the N1 assay was determined to be 1.45 gene copies per μl of reaction mixture using seven replicates of negative controls. Inhibition was not assessed for dPCR.

Viral Detection Droplet digital PCR. Droplet digital PCR was performed using the Bio-Rad QX200 system along with a C1000 Touch thermal cycler (Bio-Rad, Hercules, CA). SARS-CoV-2 RNA was detected and quantified using the N1 assay previously described. Inhibition was assessed by spiking a subset of sample extracts (n=17) with bovine respiratory syncytial virus (BRSV) RNA extracted directly from a live attenuated bovine vaccine (Inforce 3 cattle vaccine; Zoetis, Parsippany-Troy Hills, NJ) using a Qiagen PowerViral AllPrep DNA/RNA kit (Hilden, Germany). BRSV RNA was detected and quantified using an assay targeting the nucleoprotein gene with forward primer (GCA ATG CTG CAG GAC TAG GTA TAA T—SEQ ID NO: 4), reverse primer (ACA CTG TAA TTG ATG ACC CCA TTC T—SEQ ID NO: 5), and probe (FAM-ACC AAG ACT/ZEN/TGT ATG CTG CCA AAG CA-3IABkFQ—SEQ ID NO: 6) (Integrated DNA Technologies, Inc., Coralville, IA, USA) (24). Each N1 ddPCR reaction mixture was prepared as a 22-μl volume consisting of 5.45 μl of water, 5.45 μl of one-step RT-ddPCR Supermix (Bio-Rad, Hercules, CA), 2.1 μl of reverse transcriptase, 1.05 μl of dithiothreitol, forward and reverse primers at 1,000 nM, probe at 250 nM, and 5 μl of RNA extract. BRSV wells were prepared in the same manner except that forward and reverse primers at 900 nM and probe at 250 nM were used. A volume of 20 μl of each reaction mixture was passed into droplet generation. Thermal cycling was performed with reverse transcription for 60 min at 50° C., followed by 10 min at 95° C., 40 cycles of 95° C. for 30 s followed by 59° C. for 1 min, and finally 98° C. for 10 min. Each ddPCR experiment included two no-template controls each for BRSV and N1 and two positive controls each for BRSV (RNA and molecular water) and N1 (2019-nCOV_N_Positive control; Integrated DNA Technologies, Inc., Coralville, IA, USA). Manual thresholding and quantification were performed in QuantaSoft version 1.7.4 (Bio-Rad, Hercules, CA) so that no-template controls yielded no positive droplets.

The 95% limit of detection for the N1 assay was determined to be 3.3 gene copies per ddPCR reaction using a 10-replicate dilution series of synthetic SARS-CoV-2 RNA control material (catalog no. MT188340; Twist Bioscience, San Francisco, CA) with a cumulative Gaussian distribution fit to the observed proportion of the replicates positive along the dilution gradient. There was no evidence of inhibition as no difference was observed in the quantification of BRSV RNA in sample extracts compared to the BRSV positive controls (two-tailed t test, P=0.19).

Statistical and data analyses. Our goal was to compare measurement of SARS-CoV-2 in bulk dust, on surface swabs, and on a passive sampler using three different measurement methods. Each vacuum bag of dust was sampled and extracted in triplicate at each time point (immediately after collection and 1, 2, 3, and 4 weeks after collection). All three detection methods (qPCR, dPCR, and ddPCR) analyzed the same sample extractions for all sample types. Differences in positivity rates among detection methods and sample types were assessed using the Kruskal-Wallis H test. Detection limit information is described above for each detection method. The geometric mean was reported for quantification of SARS-CoV-2 RNA present in samples using each method due to the logarithmic nature of PCR-based data. Potential RNA decay over the 4-week time period was evaluated in bulk dust with a regression analysis on the ddPCR data transformed with the natural logarithm.

### REFERENCES

[1] He X, Lau E H Y, Wu P, Deng X, Wang J, Hao X, Lau Y C, Wong J Y, Guan Y, Tan X, Mo X, Chen Y, Liao B, Chen W, Hu F, Zhang Q, Zhong M, Wu Y, Zhao L, Zhang F, Cowling B J, Li F, Leung G M. 2020. Temporal dynamics in viral shedding and transmissibility of COVID-19. Nat Med 26:672-675.
[2] Rothe C, Schunk M, Sothmann P, Bretzel G, Froeschl G, Wallrauch C, Zimmer T, Thiel V, Janke C, Guggemos W, Seilmaier M, Drosten C, Vollmar P, Zwirglmaier K, Zange S, Wölfel R, Hoelscher M. 2020. Transmission of 2019-nCOV infection from an asymptomatic contact in Germany. N Engl J Med 382:970-971.
[3] Jing Q-L, Liu M-J, Zhang Z-B, Fang L-Q, Yuan J, Zhang A-R, Dean N E, Luo L, Ma M-M, Longini I, Kenah E, Lu Y, Ma Y, Jalali N, Yang Z-C, Yang Y. 2020. Household secondary attack rate of COVID-19 and associated determinants in Guangzhou, China: a retrospective cohort study. Lancet Infect Dis 20:1141-1150.
[4] van Doremalen N, Bushmaker T, Morris D H, Holbrook M G, Gamble A, Williamson B N, Tamin A, Harcourt J L, Thornburg N J, Gerber S I, Lloyd-Smith J O, de Wit E, Munster V J. 2020. Aerosol and surface stability of SARS-CoV-2 as compared with SARS-CoV-1. N Engl J Med 382:1564-1567.
[5] Aboubakr H A, Sharafeldin T A, Goyal S M. 2020. Stability of SARS-CoV-2 and other coronaviruses in the environment and on common touch surfaces and the influence of climatic conditions: a review. Transbound Emerg Dis.
[6] Kwon T, Gaudreault N N, Richt J A. 2021. Environmental stability of SARS-CoV-2 on different types of surfaces under indoor and seasonal climate conditions. Pathogens 10:227.
[7] Zhao Y, Richardson B, Takle E, Chai L, Schmitt D, Xin H. 2019. Airborne transmission may have played a role in the spread of 2015 highly pathogenic avian influenza outbreaks in the United States. Sci Rep 9:11755.
[8] Asadi S, Gaaloul Ben Hnia N, Barre R S, Wexler A S, Ristenpart W D, Bouvier N M. 2020. Influenza A virus is transmissible via aerosolized fomites. Nat Commun 11:4062.
[9] Maestre J P, Jarma D, Yu C, Siegel J, Horner S, Kinney K A. 2020. Distribution of SARS-CoV-2 RNA signal in a home with COVID-19 positive occupants. medRxiv.
[10] Bin S Y, Heo J Y, Song M-S, Lee J, Kim E-H, Park S-J, Kwon H-I, Kim S M, Kim Y-I, Si Y-J, Lee I-W, Baek Y H, Choi W-S, Min J, Jeong H W, Choi Y K. 2016. Environmental contamination and viral shedding in MERS patients during MERS-CoV outbreak in South Korea. Clin Infect Dis 62:755-760.
[11] Larsen D A, Wigginton K R. 2020. Tracking COVID-19 with wastewater. Nat Biotechnol 38:1151-1153.
[12] Ahmed W, Angel N, Edson J, Bibby K, Bivins A, O'Brien J W, Choi P M, Kitajima M, Simpson S L, Li J, Tscharke B, Verhagen R, Smith W J M, Zaugg J, Dierens L, Hugenholtz P, Thomas K V, Mueller J F. 2020. First confirmed detection of SARS-CoV-2 in untreated wastewater in Australia: a proof of concept for the wastewater surveillance of COVID-19 in the community. Sci Total Environ 728:138764.
[13] Bivins A, North D, Ahmad A, Ahmed W, Alm E, Been F, Bhattacharya P, Bijlsma L, Boehm A B, Brown J, Buttiglieri G, Calabro V, Carducci A, Castiglioni S, Cetecioglu Gurol Z, Chakraborty S, Costa F, Curcio S, de Los Reyes F L, III, Delgado Vela J, Farkas K, Fernandez-Casi X, Gerba C, Gerrity D, Girones R, Gonzalez R, Haramoto E, Harris A, Holden P A, Islam M T, Jones D L, Kasprzyk-Hordern B, Kitajima M, Kotlarz N, Kumar M, Kuroda K, La Rosa G, Malpei F, Mautus M, Mclellan S L, Medema G, Meschke J S, Mueller J, Newton R J, Nilsson D, Noble R T, van Nuijs A, Peccia J, Perkins T A, Pickering A J, Rose J, et al. 2020. Wastewater-based epidemiology: global collaborative to maximize contributions in the fight against COVID-19. Environ Sci Technol 54:7754-7757.
[14] Gonzalez R, Curtis K, Bivins A, Bibby K, Weir M H, Yetka K, Thompson H, Keeling D, Mitchell J, Gonzalez D. 2020. COVID-19 surveillance in Southeastern Virginia using wastewater-based epidemiology. Water Res 186:116296.
[15] Torrey J, von Gunten U, Kohn T. 2019. Differences in viral disinfection mechanisms as revealed by quantitative transfection of echovirus 11 genomes. Appl Environ Microbiol 85: e00961-19.
[16] Razzini K, Castrica M, Menchetti L, Maggi L, Negroni L, Orfeo N V, Pizzoccheri A, Stocco M, Muttini S, Balzaretti C M. 2020. SARS-CoV-2 RNA detection in the air and on surfaces in the COVID-19 ward of a hospital in Milan, Italy. Sci Total Environ 742:140540.
[17] Harvey A P, Fuhrmeister E R, Cantrell M E, Pitol A K, Swarthout J M, Powers J E, Nadimpalli M L, Julian T R, Pickering A J. 2021. Longitudinal monitoring of SARS-CoV-2 RNA on high-touch surfaces in a community setting. Environ Sci Technol Lett 8:168-175.
[18] Curtis L, Cali S, Conroy L, Baker K, Ou C-H, Hershow R, Norlock-Cruz F, Scheff P. 2005. Aspergillus surveillance project at a large tertiary-care hospital. J Hosp Infect 59:188-196.
[19] Faure O, Fricker-Hidalgo H, Lebeau B, Mallaret M R, Ambroise-Thomas P, Grillot R. 2002. Eight-year surveillance of environmental fungal contamination in hospital operating rooms and haematological units. J Hosp Infect 50:155-160.
[20] Zhang B Y, Liu F, Chen X D. 2015. The effect of Legionella pneumophila contamination in the surface dust of the air ducts of central air conditioning systems on indoor air quality. Int J Vent 14:231-240.
[21] Hart O E, Halden R U. 2020. Computational analysis of SARS-CoV-2/COVID-19 surveillance by wastewater-based epidemiology locally and globally: feasibility, economy, opportunities and challenges. Sci Total Environ 730:138875.
[22] Wang X, Zheng J, Guo L, Yao H, Wang L, Xia X, Zhang W. 2020. Fecal viral shedding in COVID-19 patients: clinical significance, viral load dynamics and survival analysis. Virus Res 289:198147.
[23] Centers for Disease Control and Prevention. 2020. CDC 2019-novel coronavirus (2019-nCOV) real-time RT-PCR diagnostic panel. CDC-006-00019, revision 06. Centers for Disease Control and Prevention, Atlanta, GA.
[24] Boxus M, Letellier C, Kerkhofs P. 2005. Real time RT-PCR for the detection and quantitation of bovine respiratory syncytial virus. J Virol Methods 125:125-130.
Nicholas Nastasi et al., 2021. Viability of MS2 and Phi6 Bacteriophages on Carpet and Dust, bioRxiv.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

1. A method for detection of a viral nucleic acid in a dust sample, comprising
a) extracting the viral nucleic acid from the dust sample;
b) determining a level of inhibition in the viral nucleic acid extracted from the dust sample;
c) diluting the viral nucleic acid extracted from the dust sample if the level of inhibition is increased in comparison to a control; and
d) quantifying an amount of the diluted viral nucleic acid.

2. The method of claim 1, wherein step a) comprises phenol-based lysis.

3. The method of claim 1, wherein step a) comprises using a concentration of beta-mercaptoethanol about 10 times greater than a recommended concentration.

4. The method of claim 1, wherein the amount of the diluted viral nucleic acid is quantified by a polymerase chain reaction (PCR) assay.

5. The method of claim 4, wherein the PCR assay is a quantitative reverse transcription PCR (RT-qPCR) assay.

6. The method of claim 4, wherein the PCR assay is a digital PCR assay.

7. The method of claim 6, wherein the digital PCR assay is a chip-based digital PCR (dPCR) or droplet digital PCR (ddPCR) assay.

8. The method of claim 1, further comprising sequencing the viral nucleic acid extracted from the dust sample.

9. The method of claim 1, wherein the viral nucleic acid is a viral RNA or a viral DNA.

10. The method of claim 9, wherein the viral RNA is an RNA of an airborne virus.

11. The method of claim 10, wherein the RNA of the airborne virus is a SARS-CoV-2 RNA, an influenza RNA, or a respiratory syncytial virus (RSV) RNA.

12. The method of claim 1, further comprising comparing the amount of the viral nucleic acid contained in the dust sample to a threshold value.

13. The method of claim 12, wherein the threshold value is between about 50 copies per milligram (mg) dust and about 1000 copies per mg dust.

14. The method of claim 12, wherein the threshold value is about 300 copies per mg dust.

15. A method for environmental surveillance, comprising:
collecting a dust sample from an area inside an enclosed structure;
extracting viral nucleic acid from the dust sample;
quantifying an amount of the extracted viral nucleic acid contained in the dust sample; and
determining whether a viral disease is present inside the enclosed structure based on the amount of the extracted viral nucleic acid contained in the dust sample.

16. The method of claim 15, further comprising generating a report comprising the amount of the extracted viral nucleic acid contained in the dust sample.

17. The method of claim 15, further comprising comparing the amount of the extracted viral nucleic acid contained in the dust sample to a threshold value, wherein presence of the viral disease inside the enclosed structure is determined based on the comparison.

18. The method of claim 17, wherein the viral disease is severe acute respiratory syndrome coronavirus 2 (SARS-CoV-2), and wherein the threshold value is between about 50 copies per milligram (mg) dust and about 1000 copies per mg dust.

19. The method of claim 18, wherein the threshold value is about 300 copies per mg dust.

20. The method of claim 17, further comprising recommending an action item from a risk management plan based on the comparison.

21. The method of claim 20, wherein the action item is to test individuals for the viral disease.

22. The method of claim 20, wherein the action item is to sequence viral nucleic acid extracted from the dust sample to detect variants.

23. The method of claim 15, further comprising sequencing viral nucleic acid extracted from the dust sample to detect variants.

24. The method of claim 15, further comprising correlating the amount of the extracted viral nucleic acid contained in the dust sample to an approximate number of infected individuals using the enclosed structure.

25. The method of claim 15, further comprising sieving the dust sample prior to extracting viral nucleic acid from the dust sample.

26. The method of claim 15, wherein the dust sample is a bulk dust sample.

27. The method of claim 15, wherein the dust sample is a surface swab sample or an air sample.

28. The method of claim 15, wherein the amount of the extracted viral nucleic acid from the dust sample is quantified by a polymerase chain reaction (PCR) assay.

29. The method of claim 28, wherein the PCR assay is a quantitative reverse transcription PCR (RT-qPCR) assay.

30. The method of claim 28, wherein the PCR assay is a digital PCR assay.

31. The method of claim 30, wherein the digital PCR assay is a chip-based digital PCR (dPCR) or droplet digital PCR (ddPCR) assay.

32. The method of claim 15, wherein the viral disease is severe acute respiratory syndrome coronavirus 2 (SARS-CoV-2).

33. The method of claim 15, wherein the viral nucleic acid is a viral RNA or a viral DNA.


SEQUENCE LISTING (6 sequences)

SEQ ID NO: 1 - DNA, 20 bp, Artificial Sequence
  2019-nCoV_Nl forward primer
gaccccaaaatcagcgaaat

SEQ ID NO: 2 - DNA, 24 bp, Artificial Sequence
  2019- nCov_Nl reverse primer
tctggttactgccagttgaatctg

SEQ ID NO: 3 - DNA, 21 bp, Artificial Sequence
  2019-nCoV_Nl probe
  misc_feature (1)..(1): 6-carboxyfluorescein
  misc_feature (9)..(10): ZEN
  misc_feature (21)..(21): 3IABkFQ
accccgcattacgtttggacc

SEQ ID NO: 4 - DNA, 25 bp, Artificial Sequence
  Forward primer
gcaatgctgcaggactaggtataat

SEQ ID NO: 5 - DNA, 25 bp, Artificial Sequence
  Reverse primer
acactgtaattgatgaccccattct

SEQ ID NO: 6 - DNA, 26 bp, Artificial Sequence
  Probe
  misc_feature (1)..(1): 6-carboxyfluorescein
  misc_feature (9)..(10): ZEN
  misc_feature (26)..(26): 3IABkFQ
accaagacttgtatgctgccaaagca